(19) United States
(12) Patent Application Publication
(10) Pub. No.: US 2005/0060794 A1
(43) Pub. Date: Mar. 24, 2005

(54) Protective respirator with shield attachments

(76) Inventors: Klaus-Dieter Dahrendorf, Berlin (DE); Michael Horn, Berlin (DE); Detlef Kielow, Berlin (DE); Wolfgang Weber, Berlin (DE)

Correspondence Address: Wood Phillips Katz;Clark & Mortimer, Suite 3800, 500 West Madison Street, Chicago, IL, 60661, US

(21) Appl. No.: 10/494,023

(22) PCT Filed: Sep. 20, 2002
(86) PCT No.: PCT/DE02/03639

(30) Foreign Application Priority Data
Oct. 29, 2001 (DE) 101 54 683.1

(51) Int. Cl. A61F 9/02

(52) U.S. Cl. 2/432

(57) ABSTRACT

In a respirator mask in which the visor panels (1) can be covered by light-absorbing supplementary panels (5), first (7) and second (8) snap-in hooks are molded onto opposite sides of the visor panel and a stop (6) is molded onto a corner section (3) to form a guide pocket (4). The outer contour lines and the convexity of the visor panel and the supplementary panel are identical. To attach the flexurally elastic supplementary panel (5), it is first fastened in the guide pocket (4) and on the first snap-in hook (7). Then the opposite side is locked into the second snap-in hook (8) after flexing the supplementary panel. The supplementary panel system has a low overall height, is simple and cost-efficient. Attachment is simple.

This invention relates to respirator masks with supplementary panels that can be attached as light filters in front of the visor panels provided in the facepiece.

Visor panels are inserted at the wearer's eye level into the respirator facepiece that is made of a flexible material. The practice to attach supplementary panels made of a light-filtering material in front of the visor panels, if required, to protect the eyes against light rays that can damage them or impair vision by dazzling effects is known. Mask bodies typically feature a frame around the perimeter of the visor panels for attaching supplementary panels while these supplementary panels are embedded in a rubber collar around their rims. Attaching the supplementary panels requires fitting the rubber collar onto the frame and is a tedious and time-consuming activity that becomes even more difficult when the wearer has to wear protective gloves. Moreover, the eyes must be protected immediately to prevent damage in some applications involving rays of light, which cannot be done in the required time frame without the help of others when using the known supplementary panel systems. In addition to the complex design involving a frame and a collar, another disadvantage of the supplementary panels and their attachment systems in use is that they take considerable space and make reading instrument displays very difficult.

It is therefore the problem of this invention to design a respirator mask and temporarily attachable supplementary panels for light filtering in such a way that when in use, i.e. when someone is wearing the mask, the wearer can attach supplementary panels fast and without requiring help from others and that the attachment system is to protrude from the facepiece as little as possible.

This problem is solved according to the invention by the respirator mask with supplementary panels comprising the characteristics described in claim 1.

The dependent claims disclose advantageous improvements and useful embodiments of the invention.

The general inventive idea is, in other words, that a flexurally elastic supplementary panel that is identical with the visor panel in outer contour and convexity is attached using snap-in means integrated into opposing side rim sections of the visor panel, and that a guide element is molded onto the visor panel for fast and correct positioning of the attachment. To attach the supplementary panel, it is first aligned on the visor panel using the guide element, then locked into the first snap-in means and finally locked completely into the second snap-in means after flexing it slightly so that it sits close to the visor panel.

Both the visor panel and the supplementary panel have a simple design and can be produced at low cost. The supplementary panels can be attached in no time while wearing the respirator, without requiring tools or help from others. The supplementary panel is fastened securely but it is also no problem to take it off. Another advantage is its small space requirement; the overall height of the visor area is just increased by the thickness of the supplementary panel.

The guide element is formed by a stop that is molded to a corner area of the rim of the visor panel and defines a guide pocket. The snap-in means are two snap-in hooks molded to two opposite rim sections of the visor panel and consisting of a vertical web and a horizontal holding web. Recesses are molded into the supplementary panel where the panel would be under the snap-in hook when attached so that the upper edge of the snap-in hooks is flush with the surface of the supplementary panel.

According to another characteristic of the invention, the supplementary panel is concave towards the visor panel so the slight flexing required for attachment and removal is quite simple. A recess is shaped into the rim area of the supplementary panel to provide a fingerhold for flexing the panel. This makes it easier to handle the supplementary panel during attachment and removal.

An embodiment of the invention is explained in greater detail below with reference to the figures. Wherein:

FIG. 1 shows a perspective top view of a visor panel and a partially attached supplementary panel shortly before snapping it into the visor panel;

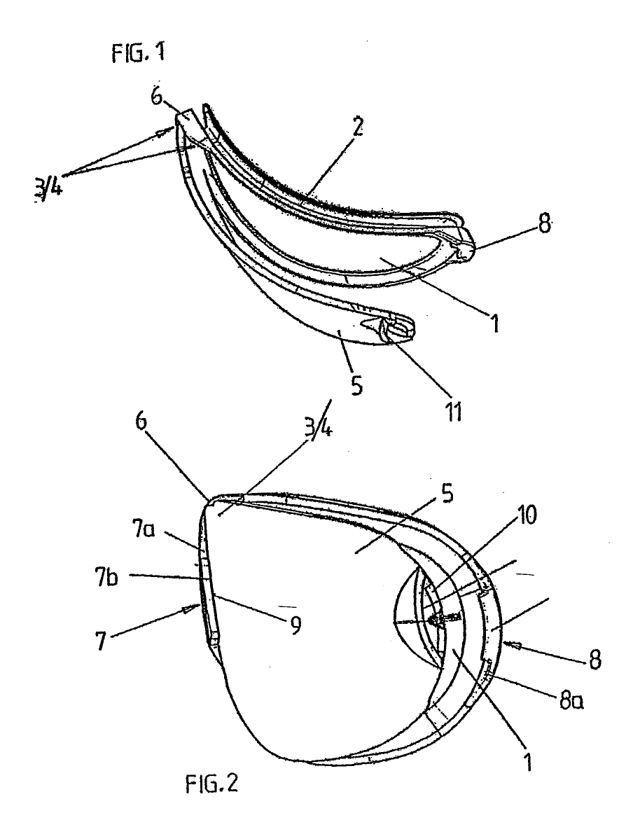

FIG. 2 shows a front view of the visor and supplementary panel system according to FIG. 1;

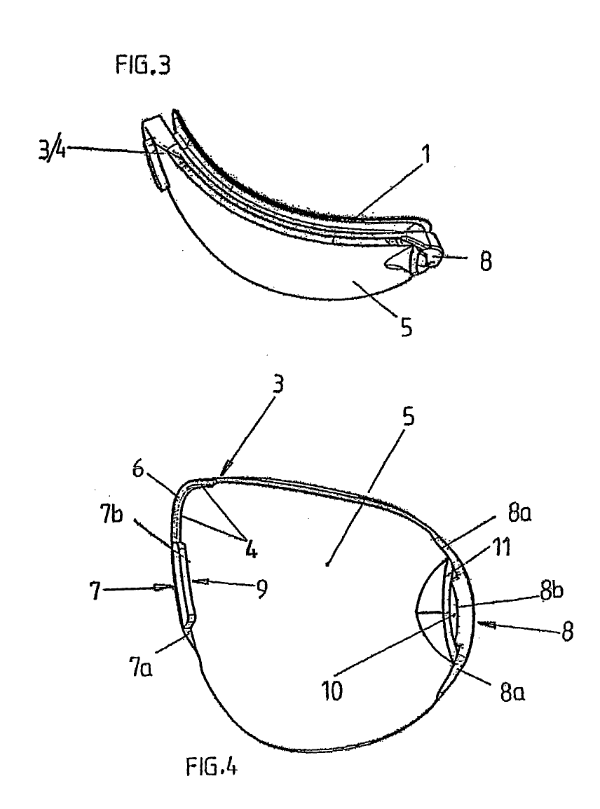

FIG. 3 shows a top view of the visor and supplementary panel system with the supplementary panel fully attached; and

FIG. 4 shows a front view of the system according to FIG. 3.

The visor panel 1 is convex and comprises a circumferential groove 2 through which it is connected with a respirator mask (not shown).

A guide pocket 4 is formed in an angular rim section 3 of the visor panel 1 for correct positioning of the supplementary panel 5 on the visor panel 1. The guide pocket 4 is formed by a stop 6 that protrudes vertically from the visor panel surface. First and second snap-in hooks 7 and 8, each of which consisting of a vertical web 7a or 8a stretching from the visor panel surface and a holding web 7b or 8b pointing in horizontal direction towards the center of the visor panel, are molded to opposite rim sections of the visor panel 1. It is apparent from FIG. 4 that the stop 6 merges with the web 7a of the first snap-in hook 7.

The shape of the supplementary panel 4 is similar to the visor panel 1 in outer contour lines and convexity. First and second recesses 9, 10 (reductions in material) that are slightly longer and wider than the horizontal webs 7b or 8b of the respective snap-in hook are molded into the supplementary panel 5 in the rim section where the supplementary panel is located under the first snap-in hook 7 and the second snap-in hook 8. A fingerhold 11 is formed in front of the second recess 10 on the supplementary panel 5. The supplementary panel 5 can be slightly flexed into one direction due to its convex design.

The supplementary panel 5 is attached to the visor panel 1 as follows:

The supplementary panel 5 is inserted with its angular (pointed) side into the guide pocket 4 until it rests against the stop 6 and thus becomes automatically aligned with the visor panel 1. The first recess 9 in the supplementary panel 5 slides under the first snap-in hook 7 so that the supplementary panel 5 is locked in on one side of the visor panel 1. As the wearer presses a finger onto the fingerhold 11 in the direction of the arrow in FIG. 2, the supplementary panel 5 is flexed somewhat more so that the second recess in the supplementary panel 5 becomes located under the second snap-in hook 10, then the supplementary panel 5 stretches due to its intrinsic elasticity and the second recess 10 slides under the second snap-in hook 8. Thus the supplementary panel 5 is securely locked in vertical direction to the visor panel surface but fixed in all lateral horizontal directions due to the curved shape of the holding web 8a and the stop 6. The supplementary panel 5 can easily be detached from the visor panel 1 when the wearer presses in lateral direction on the fingerhold 11; the supplementary panel 5 is thus flexed somewhat more so that it slides out of the second snap-in hook 8 and can be removed in upward direction.

The supplementary panels can be attached easily, fast and securely while wearing the respirator mask without requiring help from others due to the visor panel design with a guide pocket and stop as well as snap-in hooks on opposite rim sections of the visor panel. The supplementary panel basically rests on the visor panel so that the overall height of the attachment in front of the visor panel is determined by the thickness of the supplementary panel and thus very small.

### List of Reference Symbols

1 visor panel
2 groove
3 angular rim section
4 guide pocket
5 supplementary panel
6 stop
7 first snap-in hook
7a vertical web
7b horizontal web
8 second snap-in hook
8a vertical web
8b horizontal web
9 first recess
10 second recess
11 fingerhold

1. A respirator mask with supplementary panels that can be attached as light filters in front of the visor panels provided in the facepiece, characterized in that
a stop (6) is provided in an angular rim section of the visor panel (1) to form a guide pocket (4) together with the surface of the visor panel (1) after which a first snap-in hook (7) that emerges from the visor panel (1) follows, and
a second snap-in hook (8) is molded onto the rim section of the visor panel (1) opposite the first snap-in hook (7) wherein
the flexurally elastic supplementary panel (5) that is identical in shape with the visor panel (1) can be directly fastened to the visor panel (1).

2. The respirator mask according to claim 1, characterized in that the snap-in hooks (7, 8) consist of a vertical web (7a, 8a) that follows the contour line of the visor panel (1) and a horizontal holding web (7b, 8b).

3. The respirator mask according to claim 1, characterized in that a recess (9, 10) is molded into the rim of the supplementary panel (5) in the section that is level with the respective snap-in hook (7, 8).

4. The respirator mask according to claim 1, characterized in that the supplementary panel (5) and the visor panel (1) have a convex shape between the opposing snap-in hooks (7, 8) or recesses (9, 10), respectively.

5. The respirator mask according to claim 1, characterized in that a fingerhold (11) for flexing the supplementary panel (5) when it is locked onto the visor panel (1) or removed from it is formed into the surface of the supplementary panel (5) on the side opposite from the first snap-in hook (7) and the guide pocket (4).
